(19) United States
(12) Patent Application Publication
(10) Pub. No.: US 2014/0302587 A1
(43) Pub. Date: Oct. 9, 2014

(54) PROCESS FOR PRODUCING CELLULASES USING A FILAMENTOUS FUNGUS SUITABLE FOR A FERMENTER, HAVING A LOW VOLUMETRIC OXYGEN TRANSFER COEFFICIENT KLA

(71) Applicants: Fadhel Ben Chaabane, Paris (FR); Etienne Jourdier, Vaucresson (FR); Celine Cohen, Paris (FR); Bernard Chaussepied, Hanches (FR)

(72) Inventors: Fadhel Ben Chaabane, Paris (FR); Etienne Jourdier, Vaucresson (FR); Celine Cohen, Paris (FR); Bernard Chaussepied, Hanches (FR)

(73) Assignees: IFP ENERGIES NOUVELLES, Rueil-Malmaison Cedex (FR); AGRO INDUSTRIES RECHERCHE ET DEVELOPPEMENT, Pomacle (FR); INSTITUT NATIONAL DE LA RECHERCHE AGRONOMIQUE, Paris Cedex (FR)

(21) Appl. No.: 14/239,690

(22) PCT Filed: Aug. 2, 2012
(86) PCT No.: PCT/FR2012/000328

§ 371 Date: May 28, 2014

(30) Foreign Application Priority Data
Aug. 19, 2011 (FR) 11/02556

(51) Int. Cl. C12N 9/42

(52) U.S. Cl. CPC C12N 9/2437
USPC 435/209

(57) ABSTRACT

The present invention concerns a process for the production of cellulases using a strain of a filamentous fungus in a stirred, aerated bioreactor, comprising at least two growth steps:
the first growth step in the presence of at least one carbonaceous growth substrate in batch phase with a concentration of carbonaceous growth substrate in the range 10 to 80 g/L;
a second step for growth and production in the presence of at least one inducer carbonaceous substrate in fed batch phase in the presence of a limiting flow of carbon source in the range 50 to 140 mg per gram of cells per hour.

### FIELD OF THE INVENTION

The invention relates to a process for the production of cellulases using a filamentous fungus necessary for the enzymatic hydrolysis of lignocellulosic biomass used, for example, in processes for the production of biofuels known as second generation processes or in other processes in the chemicals, paper or textiles industry.

### PRIOR ART

The development of economically viable processes for the production of second generation biofuels is currently a “hot topic”. These fuels are produced from lignocellulosic biomass and give rise to fewer problems as regards competition with the use of agricultural land for food compared with “first generation” biofuels which are produced from sugar cane, corn, wheat or beet.

Lignocellulosic biomass is characterized by a complex structure constituted by three principal fractions: cellulose, hemicellulose and lignins. The conventional process for transforming it into ethanol comprises a number of steps. A pre-treatment can render the cellulose accessible to enzymes, namely cellulases. The enzymatic hydrolysis step can be used to transform cellulose into glucose which is then transformed into ethanol during the fermentation step, generally using the yeast Saccharomyces cerevisiae. Finally, a distillation step can separate and recover the ethanol from the fermentation must.

Various technico-economic studies have demonstrated that reducing the cost of cellulases is one of the key points in biological ethanol production processes starting from lignocellulosic substances. Currently, industrial cellulases are principally produced by a filamentous fungus, Trichoderma reesei, because of its high cellulase-secreting power. The strategy which is applied industrially is to cause the fungus to grow rapidly to a given concentration, then to induce the production of cellulases in order to maximize productivity and yield. Trichoderma reesei is strictly aerobic and growth of it results in a substantial increase in the viscosity of the medium, rendering difficult the transfer of oxygen, which is necessary for its survival. Oxygen transfer is linked to K_{L}a, which is known as the coefficient of volumetric oxygen transfer per unit volume of medium. It is the product of the coefficient K_{L} (overall O₂ exchange coefficient in m/s or m/h) and the coefficient “a” (specific exchange area per unit volume of liquid phase culture medium in m² per m³ of culture medium). K_{L}a is proportional to stirring and to aeration. In order to satisfy the biological demand for oxygen which increases during the fungus growth phase, it is necessary to increase oxygen transfer, generally accomplished by increasing stirring or the aeration flow rate. This results in an increase in the energy consumption of the process (power to motor for stirring and compressor for aeration) with an increase in operating costs as a consequence. The costs linked to stirring and aeration may represent up to 50% of the operating costs of a process for the production of cellulases.

One way of reducing the cost of enzyme production is thus to reduce the operating costs by modifying the operation of the process in order to minimize the K_{L}a required while maintaining the cellulose productivity. This also means that scale-up can be simplified as regards the dimensions of an industrial fermenter (typically 100 to 1000 m³) which becomes difficult for K_{L}a values of more than 200 h⁻¹.

The enzymes of the enzymatic Trichoderma reesei complex contain three main activity types: endoglucanases, exoglucanases and cellobiases or beta-glucosidases. Other proteins with functions or activities which are vital to hydrolysis of lignocellulosic materials are also produced by Trichoderma reesei, for example xylanases. The presence of an inducer substrate is vital to the expression of cellulolytic and/or hemicellulolytic enzymes.

Regulation of the genes for cellulases on various carbon sources has been studied in detail. They were induced in the presence of cellulose, its hydrolysis products (for example: cellobiose) or certain oligosaccharides such as lactose or sophorose (IImén et al, 1997; Appl Environ Microbiol 63: 1298-1306).

Conventional mutation genetic techniques have enabled the selection of strains of Trichoderma reesei which hyperproduce cellulases, such as the strains MCG77 (Gallo—U.S. Pat. No. 4,275,167), MCG 80 (Allen, A. L. and Andreotti, R. E., Biotechnol—Bioengi 1982, 12, 451-459 1982), RUT C30 (Montenecourt, B. S. and Eveleigh, D. E., Appl Environ Microbiol 1977, 34, 777-782) and CL847 (Durand et al, 1984, Proc Colloque SFM “Génétique des microorganismes industriels” [Genetics of industrial microorganisms]. Paris. H. HESLOT Ed, pp 39-50).

In order to obtain good enzyme productivity, industrial processes for the production of cellulases such as that described in patent FR 2 881 753, for example, are carried out in two steps:
a growth step in “batch” mode where it is necessary to supply a source of carbon that can be rapidly assimilated for growth of Trichoderma reesei, then
a “fed batch” production step using an inducer substrate such as lactose, for example, which allows the expression of cellulases and secretion into the culture medium. The continuous supply of such soluble carbonaceous substrates, thereby limiting the residual concentration in the cultures and thereby optimizing the quantity of sugar means that high enzymatic productivities can be obtained. The optimal flow of carbonaceous source applied in the cited patent is in the range 35 to 45 mg (of sugar).g(cellular dry weight)⁻¹.h⁻¹.

This protocol suffers from the disadvantage of necessitating a high energy output in order to satisfy the microorganism's demand for oxygen. At the end of the growth phase, the oxygen demand is very high. In fact, when all of the substrates are available in excess, growth occurs at a growth rate close to the maximum growth rate for the strain. The biological demand for oxygen, which is a function of the growth rate and the concentration of biomass, will increase. Since cultures regulate the concentration of dissolved oxygen to a constant value, the oxygen transfer rate OTR must be equal to the rate of oxygen consumption ROC (or biological demand for oxygen).

k_{L}a*(O₂*−O_{2L})=(μ*X)/R_{x/O2}

where:

O₂*: saturated oxygen concentration

O_{2L}: concentration of oxygen in the liquid phase

μ: rate of growth of microorganism (h⁻¹)

R_{X/O2}: yield of cellular biomass with respect to oxygen consumed

X=concentration of cellular biomass (dry weight)

This type of process thus requires a k_{L}a which is very high in order to satisfy this demand. This is generally accomplished by increasing stirring (or aeration), consuming electrical energy.

If the k_{L}a is halved, the maximum biomass which it is possible to obtain with the same growth rate is halved. It is possible to obtain the same quantity of biomass with a lower growth rate, but this requires more time and thus causes a drop in the final productivity for secreted enzymes.

The present invention can be used to better control the biological demand for oxygen without reducing the enzyme productivity. This is made possible by exploiting the physiological characteristics of filamentous fungi such as Trichoderma reesei under limiting conditions as regards the carbonaceous substrate.

### SUMMARY OF THE INVENTION

The present invention concerns the production of cellulases by a filamentous fungus that can be used to maintain the enzyme productivity performance, by using a bioreactor with a low volumetric oxygen transfer coefficient k_{L}a.

### DESCRIPTION OF THE DRAWINGS

FIG. 1 represents a comparison of values simulated by the model using the experimental values cited in the article by Tolan and Foody (1999) obtained with another strain of Trichoderma reesei.

FIG. 2 represents the change in the concentration of biomass, proteins and k_{L}a corresponding to Example 1.

FIG. 3 represents the change in the state variables with time corresponding to Example 2.

FIG. 4 represents the change in the state variables with time corresponding to Example 3.

FIG. 5 represents the change in k_{L}a with time corresponding to Example 3.

### DETAILED DESCRIPTION OF THE INVENTION

The present invention pertains to a process for the production of cellulases using a filamentous fungus strain in a stirred, aerated bioreactor, comprising at least two steps:
the first growth step in the presence of at least one carbonaceous growth substrate in batch phase with a concentration of carbonaceous growth substrate in the range 10 to 80 g/L;
a second step for growth and production in the presence of at least one inducer carbonaceous substrate in fed batch phase in the presence of a limiting flow of carbon source in the range 50 to 140 mg per gram of cells per hour.

Preferably, the concentration of carbonaceous growth substrate is in the range 10 to 20 g/L. More preferably, it is in the range 12 to 17 g/L.

During the growth phase, the concentration of carbonaceous substrate is such that the growth occurs at a maximum growth rate.

Preferably, the concentration of inducer carbonaceous substrate used in the fed batch phase is in the range 70 to 100 mg per gram of cells per hour. Still more preferably, it is in the range 80 to 90 mg per gram of cells per hour.

The bioreactor used in the present invention may thus have a volumetric oxygen transfer coefficient k_{L}a in the range 40 to 180 h⁻¹, preferably in the range 40 to 150 h⁻¹.

The second step is carried out under limiting inducer carbonaceous substrate conditions with a flow which is below the maximum strain consumption capacity.

The carbonaceous growth substrate is preferably selected from lactose, glucose, xylose, residues obtained after ethanolic fermentation of monomeric sugars of enzymatic hydrolysates of cellulosic biomass and/or a crude extract of hydrosoluble pentoses deriving from pre-treatment of a cellulosic biomass.

The inducer carbonaceous substrate is preferably selected from lactose, cellobiose, sophorose, residues obtained after ethanolic fermentation of monomeric sugars of enzymatic hydrolysates of cellulosic biomass and/or a crude extract of hydrosoluble pentoses deriving from pre-treatment of a cellulosic biomass.

The inducer growth substrates cited above may be used alone or as a mixture.

Depending on its nature, the carbonaceous growth substrate selected for producing the biomass is introduced into the fermenter before sterilization or it is sterilized separately and introduced into the bioreactor after sterilization.

The inducer carbonaceous substrate introduced during the fed batch phase is sterilized independently before being introduced into the reactor.

Preferably, when the inducing carbon source is lactose, the aqueous solution is prepared in a concentration of 200-250 g/L.

The process of the present invention can be used to obtain an analogous cellulase productivity using a bioreactor with an oxygen transfer capacity which is two and a half times smaller, i.e. a K_{L}a of 100 h⁻¹ instead of 250 h⁻¹. The correlation linking K_{L}a to the power dissipated in the aerated and stirred bioreactors such as that indicated, for example, in the NREL report “Lignocellulosic biomass to ethanol process design and economics utilizing co-current dilute acid prehydrolysis and enzymatic hydrolysis, current and futuristic scenarios”, R Wooley et al. NREL/TP-580-26157 (1999), enzyme production section, is as follows:

K_{L}a=0.026*(P/V)^{0.4}*V_{G}^{0.5}

in which:

P/V: dissipated power, in W/m³

V_{G}: surface velocity of gas (m/s).

Thus, according to this correlation, the dissipated power (P/V) for a K_{L}a of 100 h⁻¹ is approximately 10 times smaller than that necessary when the K_{L}a is 250 h⁻¹.

The advantage of a process in accordance with the present invention is to allow a simplification in the scale-up of the process to an industrial scale (typically from 100 to 1000 m³) and a reduction in operating costs.

The process is simple, robust and exploits the physiological properties of the fungus under limitation conditions with the carbonaceous substrate.

Compared with the conventional process, the operating mode has been adapted on the one hand by reducing the initial concentration of growth substrate during the first phase of the “batch” mode process in order to reduce the maximum biological oxygen demand at the end of this phase, and on the other hand by increasing the flow of carbonaceous substrate during the “fed batch” phase in order to continue producing cellular biomass during the start of this phase at a reduced growth rate, at the same time as the enzymes. The physiological properties of the fungus are exploited in order to determine the flow rate of the fed batch and the desired concentration of biomass.

This means that the final productivity performance can be maintained while requiring smaller oxygen transfer capacities for the bioreactor.

The strain used in the process is a strain of a filamentous fungus belonging to the genera Trichoderma, Aspergillus, Penicillium or Schizophyllum.

Preferably, the strains employed are strains belonging to the species Trichoderma reesei.

The industrial strains used belong to the species Trichoderma reesei, possibly modified to improve the cellulolytic and/or hemicellulolytic enzymes by mutation-selection processes such as, for example, the strain IFP CL847; strains improved by genetic recombination techniques may also be used. These strains are cultured in stirred, aerated fermenters under conditions which are compatible with their growth and with enzyme production. Other microorganism strains producing enzymes using processes similar to those used for Trichoderma may be used.

Preferably, the strain used is a strain of Trichoderma reesei modified by genetic mutation, selection or recombination.

As an example, the strain is a strain of CL847, RutC30, MCG77, or MCG80.

A flow of inducer carbonaceous substrate, qs, of more than approximately 140 mg of sugar per g of biomass per h causes an accumulation of sugar in the medium and modifies the physiological behaviour of the Trichoderma reesei, resulting in a fall in the specific rate of protein production, qp (catabolic repression phenomenon). By limiting this flow to values in the range 50 to 140 mg of sugar per gram of biomass per hour, when under limitation conditions, the strain simultaneously produces biomass and enzymes and tends towards an equilibrium state where it makes only enzymes.

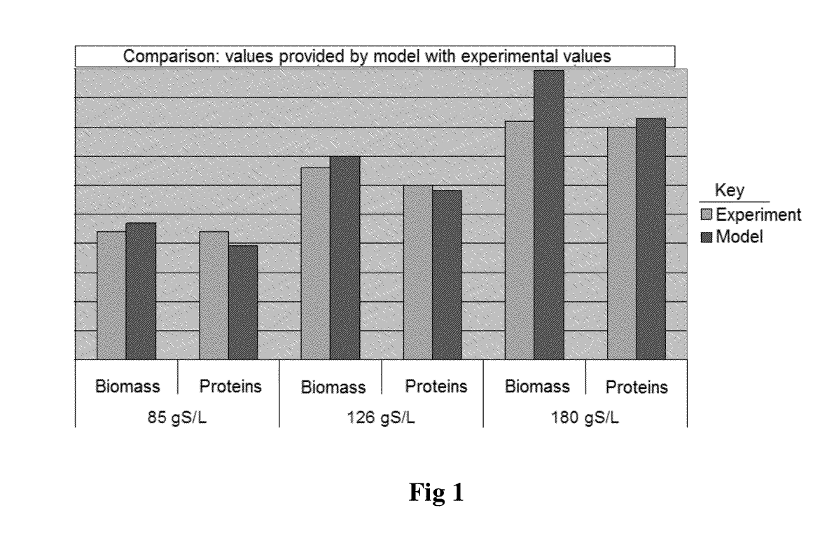

A model was established on the basis of these observations and was applied to simulate the production of biomass and enzymes carried out continuously at a degree of dilution of 0.018 h⁻¹ for various initial concentrations of carbonaceous substrate. The conditions correspond to the studies described in the article by Tolan and Foody (“Cellulase from submerged fermentation”, (1999), Adv in biochemical engineering biotechnology, Vol 65, p42-67) which cites the work by Nicholson et al. (1989) (Proceedings of the 7th Canadian bioenergy seminar, Energy Mines, and Resources) and provides experimental results for the concentrations of biomass and proteins. We retained the kinetic parameters identified for the CL847 strain even though those authors used a different strain. The results are reported in FIG. 1 and demonstrate a very good agreement between the model and the experiment.

This also demonstrates that this model is valid for other strains of Trichoderma reesei which have the same physiological behaviour as the CL847 strain.

The concentration of carbonaceous growth substrate during the batch phase was lower compared with the prior art disclosure (FR 2 881 753) in order to reduce the maximum biological demand for oxygen at the end of this phase (at μmax) for a bioreactor with a k_{L}a of 100 h⁻¹. The flow of carbonaceous substrate was then increased during the “fed batch” phase compared with patent FR 2 881 753 which recommended a flow in the range 35 to 45 mg of inducer carbonaceous substrate per gram of biomass per hour. This meant that biomass could be continued to be produced at the same time as the enzymes during the start of this phase, but at a reduced growth rate, which meant that the biological demand for oxygen could be controlled. The flow of carbon source during the fed batch phase was thus increased to a value of more than 50 mg of sugar per gram of biomass per hour at the start of the fed batch phase. Growth continued at the same time as enzyme production and stabilized when the flow of carbonaceous source was close to optimal for the strain.

### EXAMPLES

In the examples below, Example 1 presents a culture using the reference conditions of patent FR 2 881 753 with a bioreactor with a k_{L}a of 250 h⁻¹. Example 2 presents an experiment carried out under the same conditions as Example 1 with a fermenter with a k_{L}a of 100 h⁻¹. This example ended with an accumulation of carbonaceous substrate with a high biomass production and a low enzyme production. Example 3 was that implementing the process of the present invention. It was used to obtain a productivity analogous to that of Example 1 with a bioreactor with a k_{L}a of 100 h⁻¹.

### Example 1

### Production of Enzymes on Glucose

Cellulose production was carried out in a mechanically stirred fermenter. The mineral medium had the following composition: KOH 1.66 g./L, 85% H₃PO₄ 2 mL/L, (NH₄)₂SO₄ 2.8 g/L, MgSO₄, 7 H₂O 0.6 g/L, CaCl₂ 0.6 g/L, MnSO₄ 3.2 mg/L, ZnSO₄, 7 H₂O 2.8 mg/L, CoCl₂ 10 4.0 mg/L, FeSO₄, 7 H₂O 10 mg/L, Corn Steep 1.2 g/L, anti-foaming agent 0.5 mL/L.

The fermenter containing the mineral medium was sterilized at 120° C. for 20 minutes, the carbonaceous source was a solution of glucose sterilized at 120° C. for 20 minutes then added to the fermenter in a sterile manner in order to produce a final concentration of 30 g/L. The fermenter was seeded to 10% (v/v) with a liquid preculture of the Trichoderma reesei CL847 strain. The mineral medium for the preculture was identical to that of the fermenter apart from the addition of 5 g/L of potassium phthalate to buffer the pH. The growth of the fungus during preculture was carried out using glucose as the carbonaceous substrate, at a concentration of 30 g/L. Growth of the inoculum lasted 2 to 3 days and was carried out at 28° C. in a stirred incubator at atmospheric pressure. Transfer to the fermenter was carried out if the residual concentration of glucose was less than 15 g/L.

The experiment carried out in the bioreactor comprised two phases:
a phase for growth on a glucose carbonaceous substrate (initial concentration=30 g/L) at a temperature of 27° C. and a pH of 4.8 (set using a 5.5 M ammoniacal solution). Aeration was at 0.5 vvm and stirring was increased to between 200 and 800 rpm as a function of the O₂ pressure (pressure of dissolved oxygen), which was set at 30%.
an enzyme production phase. After 30 hours, the 250 g/L lactose solution was continuously injected at a flow rate of 35 mg per g of cells per hour for 250 hours. The temperature was dropped to 25° C. and the pH to 4 until culture had ended. The pH was set by adding a 5.5 M ammoniacal solution which provided the nitrogen necessary for synthesis of the excreted proteins. The quantity of dissolved oxygen was kept above 30% by the stirring action.

Enzyme production was followed by assaying the extracellular proteins using Lowry's method based on a calibration carried out with BSA (“Bovine serum albumin”), after separation of the mycelium by filtering or centrifuging. The cellulolytic activities determined were as follows:
the filter paper activity or FP, expressed in FPU (filter paper units) which could be used to assay the overall activity of the endoglucanases and exoglucanases enzymatic pool;
the β-glucosidase and xylanases activities for the specific activities.

The FP activity was measured on Whatman N^{o} 1 paper (procedure recommended by the IUPAC biotechnological commission) at an initial concentration of 50 g/L; the test sample of the enzymatic solution to be analyzed which liberated the equivalent of 2 g/L of glucose (colorimetric assay) in 60 minutes was determined. The principle of filter paper activity is to determine, by DNS (dinitrosalicylic acid) assay, the quantity of reducing sugars obtained from a Whatman N^{o} 1 paper.

The substrate used to determine the β-glucosidase activity was p-nitrophenyl-β-D-glucopyranoside (PNPG). It is cleaved by β-glucosidase, which liberates p-nitrophenol.

One β-glucosidase activity unit is defined as the quantity of enzyme necessary to produce 1 μmole of p-nitrophenol from PNPG per minute and is expressed in IU/mL.

The principle of assaying the xylanase activity resides in determining, by DNS assay, the quantity of reduced sugars obtained from the hydrolysed xylane solution. This assay method uses the reducing properties of sugars, principally xylose. The xylanase activity is expressed in IU/mL and corresponds to the quantity of enzyme necessary to produce 1 μmole of xylose per minute.

The specific activities are obtained by dividing the activities expressed in IU/mL by the concentration of proteins. They are expressed in IU/mg.

The K_{L}a values were determined from gas balances after verifying the carbon and redox balances.

The analytical determinations on the final must of Example 1 produced the following results:

Cellular biomass, g/L=15.5
Proteins, g/L=50.1
Productivity=0.20 g/L/h

### FPU=29.1 IU/mL

Specific xylanase=8.2 IU/mg
Specific β-glucosidase=1.0 IU/mg

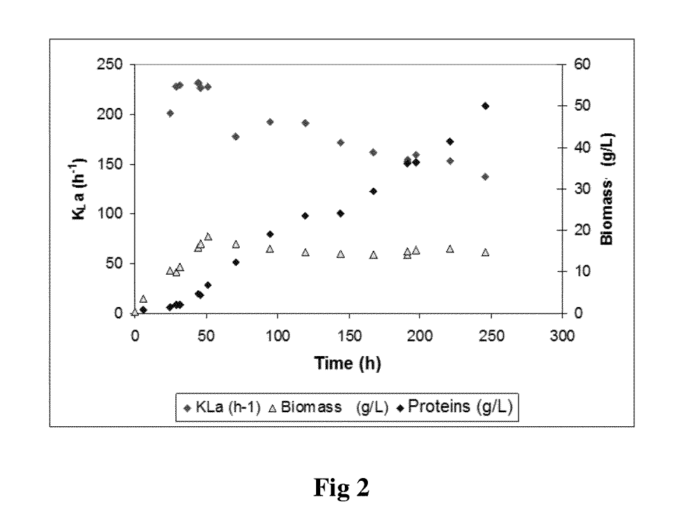

FIG. 2 repeats the change in the concentration of biomass, proteins and K_{L}a over time. It will be seen that the cellular biomass increased up to 15 g/L during the first 50 hours of the experiment and that the K_{L}a was 240 h⁻¹. The concentration of proteins increased slightly during the first 50 hours then greatly from the moment at which the concentration of biomass was stable. It reached 50 g/L when the culture had been completed.

### Example 2

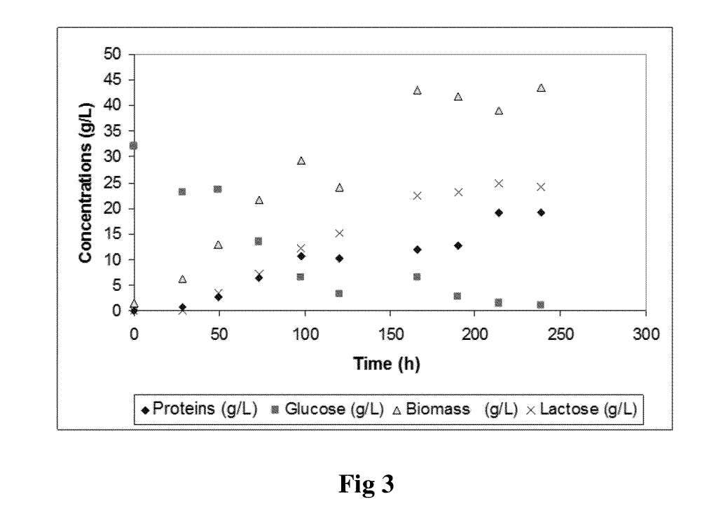

Example 2 was implemented under the same conditions as Example 1, except that the fermenter used had a maximum K_{L}a of 100 h⁻¹ and its initial volume was 750 mL. Fermentation resulted in high production of cellular biomass (45 g/L) and low protein production (19 g/L) (see FIG. 3).

This was due to the presence of large residual concentrations of carbonaceous substrate (lactose, glucose) throughout the experiment. This restarted cellulose production, which was induced under limitation conditions for the inducer carbonaceous substrate. The glucose was not consumed as the oxygen transfer capacities of the bioreactor were 2.5 times smaller, which reduced the rate of carbonaceous substrate consumption which was proportional to the rate of O₂ consumption.

In fact, the oxygen transfer rate is expressed as follows:

OTR=K_{L}a (O₂*−O_{2L})

in which:

O₂*: saturated oxygen concentration

O_{2L}: concentration of oxygen in the liquid phase

The rate of oxygen consumption was thus limited by the OTR which was 2.5 times smaller.

The analytical determinations on the final must produced the following results:

Cellular biomass, g/L=45
Proteins, g/L=19

### FPU=10.1 IU/mL

Specific xylanase=8.5 IU/mg
Specific β-glucosidase=1.2 IU/mg

### Example 3 (in Accordance with the Invention):

The bioreactor with a k_{L}a of 100 h⁻¹ was used, but the production process was modified in order to be in accordance with the present invention.

The initial glucose concentration was thus reduced to 15 g/L so that the maximum biological rate of dioxygen consumption was compatible with the fermenter used. The yield of cellular biomass production with respect to glucose was 0.5 g/g when this was present in excess. This means that the maximum dioxygen consumption rate for this quantity of glucose was 0.5 g/L/h (for a maximum growth rate of 0.08 h⁻¹ and a dioxygen to biomass conversion yield of 1.2 g/g).

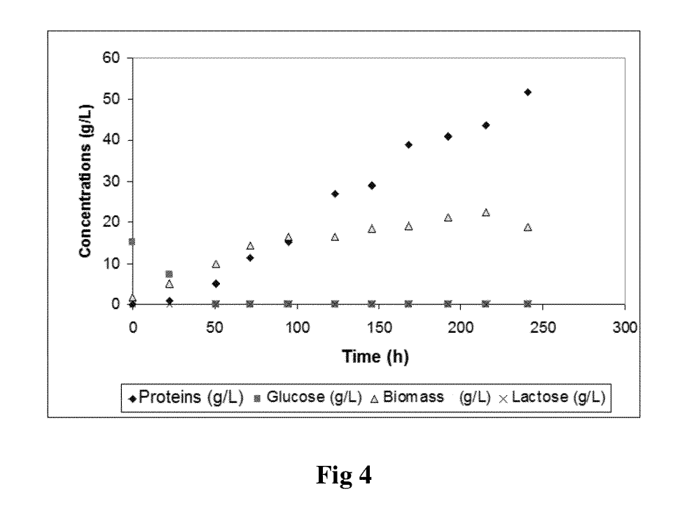

The fed batch phase was launched after 24 hours with a flow of 89 mg of substrate per gram of biomass per hour (250 g/L lactose solution). The growth phase occurred at the same time as protein production. The latter reached 51.7 g/L after 240 hours of experimentation (FIG. 4). The final protein productivity was thus maintained, despite the use of a fermenter having reduced transfer capacities. It was 0.21 g/L/h (it was 0.20 g/L/h in the case of Example 1).

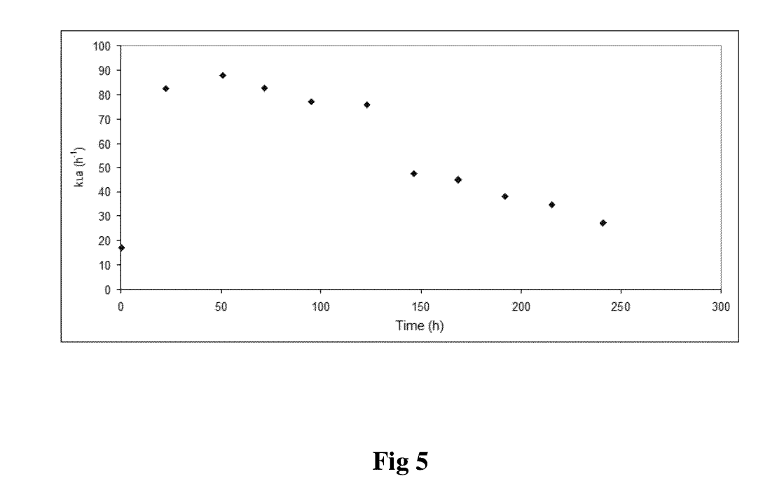

FIG. 5 illustrates the change in k_{L}a during the experiment; it stayed below 100 h⁻¹.

The analytical determinations on the final must gave the following results:

Cellular biomass, g/L=18.9
Proteins, g/L=51.7
Productivity=0.21 g/L/h

### FPU=30.1 IU/mL

Specific xylanase=9.5 IU/mg
Specific β-glucosidase=1.12 IU/mg.

1. A process for the production of cellulases using a strain belonging to a filamentous fungus in a stirred, aerated bioreactor, comprising at least two steps:
the first growth step in the presence of at least one carbonaceous growth substrate in batch phase, carried out with a concentration of carbonaceous growth substrate in the range 10 to 60 g/L;
a second step for growth and enzyme production in the presence of at least one inducer carbonaceous substrate in fed batch phase in the presence of a limiting flow of carbon source in the range 50 to 140 mg per gram of cellular biomass per hour.

2. A process according to claim 1, in which the concentration of carbonaceous growth substrate is in the range 10 to 20 g/L.

3. A process according to claim 2, in which the concentration of carbonaceous substrate is in the range 12 to 17 g/L.

4. A process according to claim 1, in which the flow of the carbon source is in the range 70 to 100 mg per gram of cellular biomass per hour.

5. A process according to claim 4, in which the flow of the carbon source is in the range 80 to 90 mg per gram of cellular biomass per hour.

6. A process according to claim 1, in which the bioreactor has a volumetric oxygen transfer coefficient, k_{L}a, in the range 40 to 180 h⁻¹.

7. A process according to claim 1, in which the bioreactor has a volumetric oxygen transfer coefficient, k_{L}a, in the range 40 to 150 h⁻¹.

8. A process according to claim 1, in which the carbonaceous growth substrate is selected from lactose, glucose, xylose, residues obtained after ethanolic fermentation of monomeric sugars of enzymatic hydrolysates of cellulosic biomass and/or a crude extract of hydrosoluble pentoses deriving from pre-treatment of a cellulosic biomass.

9. A process according to claim 1, in which the inducer carbonaceous substrate is lactose, cellobiose, sophorose, residues obtained after ethanolic fermentation of monomeric sugars of enzymatic hydrolysates of cellulosic biomass and/or a crude extract of hydrosoluble pentoses deriving from pre-treatment of a cellulosic biomass.

10. A process according to claim 1, in which the carbonaceous growth substrate selected for producing biomass is introduced into the fermenter before sterilization.

11. A process according to claim 1, in which the carbonaceous growth substrate selected for producing the biomass is sterilized separately and introduced into the bioreactor after sterilization.

12. A process according to claim 1, in which the inducer carbonaceous substrate introduced during the fed batch phase is sterilized independently before being introduced into the reactor.

13. A process according to claim 1, in which the strain used is a strain of Trichoderma reesei.

14. A process according to claim 1, in which the strain used is a strain of Trichoderma reesei modified by genetic mutation, selection or recombination.
